(19) United States
(12) Patent Application Publication
(10) Pub. No.: US 2003/0145853 A1
(43) Pub. Date: Aug. 7, 2003

(54) Expiration- dependent gas dosage

(76) Inventor: Rainer Muellner, Wiener (AT)

Correspondence Address: Connolly Bove Lodge & Hutz, P O Box 2207, Wilmington, DE, 19899-2207, US

(21) Appl. No.: 10/149,615

(22) PCT Filed: Dec. 6, 2000
(86) PCT No.: PCT/EP00/12244

(30) Foreign Application Priority Data
Dec. 15, 1999 (DE) 199604045

(51) Int. Cl. A61M 16/00

(52) U.S. Cl. 128/204.18; 128/204.21; 128/204.22

(57) ABSTRACT

The gas-supply system entailing controlled dosing of at least one gas or at least one aerosol is characterized by a control means for a dosing whereby the feed of the gas or aerosol into a breathing gas starts at a defined point in time during the expiration of a patient.

The invention relates to a gas-supply system entailing controlled dosing of at least one gas or at least one aerosol, it also relates to its use and to a method for dosing gas to supply humans or animals with one or more gases within the scope of inhalation treatment,

Breathing devices are employed in mechanical ventilation, anesthesia and respiratory therapy calling for treatment with gases such as, for instance, oxygen administration or treatment with nitric oxide (NO).

Patients suffering from chronic breathing difficulties (for example, asthma or chronic obstructive pulmonary disease—COPD) use a normally portable oxygen dispenser to supply oxygen to the body. Such patients are referred to as spontaneously breathing patients, in contrast to patients who are intubated and hooked up to a ventilator in a hospital. Spontaneously breathing patients are given, for example, additional oxygen (LOT=long-term oxygen therapy) or breathing support (via continuous positive airways pressure—CPAP). The gases are administered either via so-called nasal clips or nasal probes (nasal administration; in the simplest case, a gas-supply tube whose opening is positioned open below the nostrils of the patients) or via a breathing mask (especially in the case of CPAP).

WO 98/31282 (internal designation TMG 2028167), describes a gas-supply system for ventilated or spontaneously breathing patients with which one or more gases (for example, NO, oxygen) are dosed irregularly (continuously or discontinuously) into the breathing gas by a control means (program control, sensor control or combined program-sensor control).

With the known gas-supply systems, the dosing of the gas is inspiration-triggered.

An important aspect in triggering the gas dosing is the maximum value of the inspiratory flow, since normally the dosed gas should already be available at this point in time. As a rule, this maximum value coincides with the triggering starting point. Owing to mechanical, electrical but especially flow-related delays, the beginning cannot occur simultaneously with the gas flow that is actually being dosed into the nasopharyngeal cavity at the maximum inspiratory flow. Particularly the dead space volume plays an important role here. In addition to the nasopharyngeal cavity, the anatomical dead space encompasses the trachea, bronchi and bronchioles. In adults, this dead space amounts to between 150 mL and 200 mL. Moreover, some of the breathing gas that reaches the alveoli is not utilized due to diminished perfusion of the alveoli in question. This dead space is referred to as alveolar dead space. This value can vary widely from patient to patient. COPD) patients usually have a higher respiration rate coupled with a smaller tidal volume. Assuming a tidal volume of 400 mL and a dead space volume of 200 mL, it can be seen that the dead space volume equals 50% of the breathing volume. This greatly impairs the therapeutic effect.

Therefore, when it comes to inhalation therapy, the gas should be administered in such a way that, to the greatest extent possible, the entire amount is available at the site of action, namely, the alveolar area.

The invention is based on the objective of optimizing gas dosing in inhalation therapy, especially for spontaneously breathing patients.

This objective is achieved by means of a gas-supply system having the features described in claim 1.

The expiration-triggered gas-supply system according to the invention is based on a gassupply system for ventilated or spontaneously breathing patients as described, for example, in WO 98/31282 (internal designation TMG 2028/67), to which reference is hereby made. The gas-supply system described in WO 98/31282 is advantageously modified, as will be explained below.

The gas-supply system is employed for humans and animals, especially mammals.

The effect of the bolus (“gas package”) is utilized go that a higher concentration is available at the site of action (much higher than the average concentration), since the homogenization takes a certain amount of time. Consequently, there is also a higher partial pressure differential, which results in a higher diffusion at the site of action (for instance, in the alveoli). For this purpose, it is necessary to know the beginning of the inspiratory phase as precisely as possible and to react to the above-mentioned effects.

In any case, the therapy gas (for instance, O₂, NO) has to be administered in such a manner that it does not remain in the dead space, that is to say, in any case, it must participate in the gas exchange or even improve it, by ensuring that the bolus reaches the site of action at the highest concentration possible.

The therapy gas is administered to the patient at a defined point in tine prior to the beginning of the inspiration in order to ensure that the gas in question actually reaches the regions of the lungs that it is supposed to reach. For this purpose, it is necessary to know the course-of the expiration in order to precisely define the starting point for the dosing. In particular, this can be ensured by measuring the pressure course during one breathing cycle (expiration and inspiration), for example, in the nasal clips, usually using a pressure sensor or a flow sensor (or a system based on these).

The pressure course varies for each patient. Since this pressure course is quite similar during each breathing cycle, it is possible to tell from a momentary expiratory pressure when the patient is going to inhale. In other words, the point in time of the beginning of the inspiration can be predicted on the basis of a threshold value of the appertaining pressure value. Here, the expiration curve for each patient is recorded and, by means of an algorithm, a certain point in time prior to inhalation is associated with each pressure value (depending on whether the curve is rising or falling). On the basis of the patient-specific curve recorded by the physician, every point in time of the expiration is precisely defined as a function of the pressure course. Consequently, the triggering pulse is not initiated by the negative pressure generated at the time of inhalation, but rather, by an adjustable positive pressure threshold value resulting from the expiration course. In the case of triggering during the expiration, the triggering is adapted to the patient's needs through the possibly fluctuating expiration course, since the triggering does not take place on the basis of a time constant but rather, on the basis of the patient-dependent positive pressure in the expiration phase. In this manner, it can be ensured that the triggering will be automatically adapted as a function of the breathing curve of the patient. In other words, when the patient is under greater exertion, which also causes the breathing curve to change, the triggering is automatically adapted to the changed conditions. As a result, the dosing of one or more gases can be controlled in such a way that various areas of the lung can be systematically exposed to the therapy gas as a function of the given individual physiology of the patient.

Furthermore, the possibility exists to dose different gases at different points in time during expiration. These points in time are precisely defined by means of the pressure curve and this makes it possible to supply different gases or gas concentrations to different areas of the lung with each breath.

This method can be advantageously employed for all gases that are suitable for the therapy of lung diseases.

Especially patients whose disease (for instance, pulmonary fibrosis) had so far made them dependent on a continuous supply of O₂ can now use this system to switch over to pulsed dosing and consequently to a lower O₂ consumption, even though the blood gas values remain at about the same level as with a continuous supply of gas.

Another area of application of the method is, for instance, a gas or aerosol therapy in the nasopharyngeal cavity or in the trachea. This means here that the site of action is not directly in the lung, but rather in the anatomical dead space.

This is likewise advantageously achieved by means of dosing that is implemented during expiration and this can be regulated precisely.

The invention will be explained with reference to the drawing.

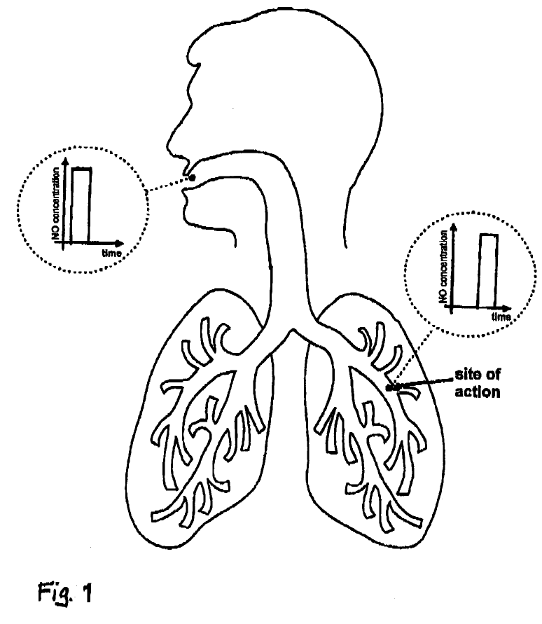

FIG. 1 shows the effect of the expiration-triggered gas dosing, whereby a gas surge (bolus) of the dosed gas reaches the site of action, for example, the lung of the patient.

FIG. 2 schematically shows an expiration curve recorded before or during the gas treatment, whereby the pressure p (in mbar) recorded by means of a sensor (for example, in front of the nose or in a breathing mask) is expressed as a function of the time t (in seconds, s). The mark a constitutes the point in time when a defined threshold value of the pressure p has been reached while the mark b indicates the point in time of the beginning of the inspiration.

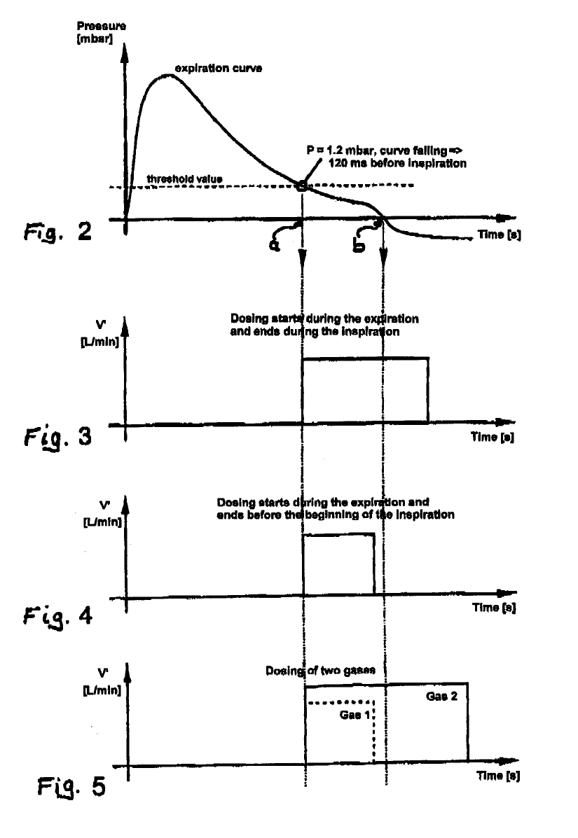

FIGS. 3 through 5 schematically show the volume flow V′ (in L/min) of dosed gas (e.g. oxygen) as a function of the time t (in seconds, s) at different dosing intervals. The gas dosing shown in FIG. 3 starts at point in time a during the expiration and ends after the beginning of the inspiration, at point in time b, during the inspiration. The gas dosing shown in FIG. 4 begins at point in time a during the expiration and ends before the beginning of the inspiration, prior to point in time b. FIG. 5 shows the dosing of two gases which combines the modes of gas dosing depicted in FIG. 4 and FIG. 3.

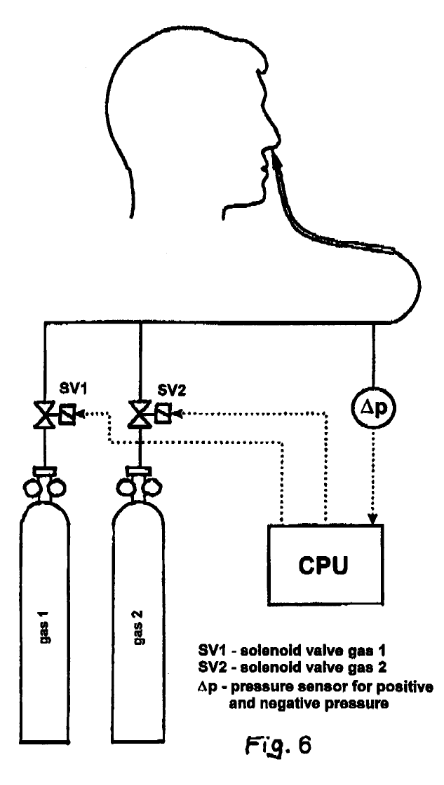

FIG. 6 shows a diagram of a gas-supply system. The gas-supply system is configured for dosing two gases (gas 1 and gas 2) which are provided, for example, in pressurized gas tanks. The gas is dosed into a gas line loading to the patient via solenoid valves (SV1 and SV2) linked to a control unit (CPU). A pressure sensor (designated with Δp) for negative and positive pressure is installed in the gas line or, for example, at the outlet of the gas line (for instance, in front of the nose of the patient).

FIG. 1 shows how a defined ratio between gas flow, dosing time and the corresponding starting point of the dosing during the expiration can be used to provide systematic therapy to any desired placed in the respiratory organs. Particularly by means of brief dosed gas surges (bolus), higher concentrations can be achieved at the site of action without adversely affecting other areas. This translates into a reduction in gas consumption—which, in turn, accounts for smaller and thus lighter storage containers—as well as into a minimization of possible side effects of the therapy. The brief time of dosing does not allow the gas mixture to become homogenized and the dosing surge propagates itself all the way to the desired site of action (FIG. 1).

An example of an expiration curve as the basis for triggering and regulating a dosing procedure is shown in FIG. 2. If, as the pressure values fall, the expiration pressure P reaches the defined value or the threshold value of 1.2 mbar determined during the ventilation, the dosing (in the example, this corresponds to a time of 120 ms prior to the beginning of the inspiration) is triggered, and then many different forms of dosing (see FIGS. 3, 4, 5) can be carried out.

By dosing the gases or aerosols only during the expiration, the anatomical dead space can systematically be exposed to the flow of gas. For example, the nasopharyngeal cavity or the trachea can be treated in a targeted manner (FIG. 3).

The dosing can be done either via a nose clip or by means of a breathing mask.

The pressure course is advantageously recorded by the same pressure sensor that is responsible for initiating the triggering signal (FIG. 6).

The dosing sequence will be explained below.

At a specific point in time of the expiration (mark a), a certain quantity of gas or aerosol is dosed. The dosing can proceed either only during the expiration (therapy in the anatomical dead space) or else during the inspiration as well (FIGS. 3, 4). Furthermore, several gases can be dosed (FIG. 5), whereby the starting point of the dosing (mark a in FIGS. 3 through 5) does not necessarily have to be the same. The dosing amounts and dosing times are greatly dependent on the therapy in question and can be varied at will.

The starting point of the dosing, the duration of the dosing as well as the dosing mount all vary as a function of the lung areas that are to be exposed to the flow.

1. An expiration-triggered gas-supply system for the gas treatment of humans and animals.

2. A gas-supply system entailing controlled dosing of at least one gas or at least one aerosol, characterized by a control means for dosing whereby the feed of the gas or aerosol into a breathing gas starts at a defined point in time during the expiration.

3. The gas-supply system according to claim 2, characterized in that the determination of the time for beginning to feed the gas or aerosol is based on a recorded breathing curve or expiration curve.

4. The gas-supply system according to claim 2 or 3, characterized in that the gas-supply system comprises at least one pressure sensor that serves to control and/or trigger the gas dosing.

5. The gas-supply system according to one of claims 1 through 3, characterized in that the gas-supply system is program-controlled or else program- and sensor-controlled and comprises a control means.

6. The gas-supply system according to one of claims 1 through 4, characterized in that the gas-supply system comprises a control means that serves to automatically adapt the triggering of the gas dosing as a function of the breathing curve of the patient.

7. A method for dosing gas to supply humans or animals with one or more gases within the scope of inhalation treatment, characterized in that the dosing of the gas is expiration-triggered.

8. The method according to claim 7, characterized in that the gas is dosed in sequences.

9. The method according to claim 7 or 8, characterized in that the gas dosing only takes place during the expiration or else the gas dosing starts during the expiration and ends during the inspiration of one breathing cycle.

10. A method to operate a gas-supply system for humans and animals, characterized in that the dosing of the gas is expiration-triggered.

11. The use of a gas-supply system according to one of claims 1 through 6 for purposes of supplying gas to ventilated or spontaneously breathing patients.

12. The use according to claim 11 for purposes of supplying gas to COPD patients.

13. The use according to claim 11 or 12, characterized in that oxygen and gas containing NO; oxygen, gas containing NO and helium; oxygen, gas containing NO, carbon dioxide and helium; oxygen, carbon dioxide and helium; or oxygen, gas containing NO and hydrogen are dosed.
